(19) United States
(12) Patent Application Publication
(10) Pub. No.: US 2006/0083793 A1
(43) Pub. Date: Apr. 20, 2006

(54) Nutritional composition for promoting muscle performance and acting as hydrogen (H+) blocker

(76) Inventors: Paul T. Gardiner, Oakville (CA); Marvin A. Heuer, Toronto (CA)

Correspondence Address: KENYON & KENYON LLP, ONE BROADWAY, NEW YORK, NY, 10004, US

(21) Appl. No.: 11/240,267

(22) Filed: Sep. 29, 2005

Related U.S. Application Data
(60) Provisional application No. 60/614,134, filed on Sep. 29, 2004.

(51) Int. Cl. A61K 36/258; A61K 36/185; A61K 31/70; A61K 31/522; A61K 31/185; A61K 31/7076

(52) U.S. Cl. 424/641; 424/725; 424/728; 424/646; 424/655; 514/114; 514/23; 514/283; 514/554; 514/553; 514/263.34; 514/440; 424/702; 514/565; 514/356; 514/546; 514/47; 514/474; 514/560

(57) ABSTRACT

A nutritional composition that includes creatinol, e.g., creatinol O-phosphate, wherein the nutritional composition includes a timed release delivery system. The timed release delivery system may include one of quick release, slow release and controlled release delivery system. Ingestion of the nutritional composition provides a method for, e.g., promoting muscle performance and/or acting as an hydrogen (H+) blocker.

### RELATED APPLICATIONS

This application is related to and claims the benefit of priority to U.S. Provisional Patent Application Ser. No. 60/614,134, entitled “Nutritional Composition for Promoting Lean Muscle Mass and Enhancing Muscle Performance and Endurance,” filed on Sep. 29, 2004, the disclosure of which is hereby fully incorporated by reference herein.

### FIELD OF THE INVENTION

The present invention relates to a nutritional composition for promoting muscle performance and acting as a hydrogen (H+) blocker, a method of providing same by consuming the nutritional composition and a method of manufacturing such a nutritional composition.

### SUMMARY OF THE INVENTION

The present invention provides for a nutritional composition including at least creatinol, which provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. Still further, the nutritional composition may provide, for a person suffering from amyotrophic lateral sclerosis (“ALS”), also referred to as Lou Gehrig's disease, an improvement in the symptoms associated with that disease.

The present invention also provides, by the consumption of the nutritional composition, a method for provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output.

The present invention also provides, in some embodiments, for a timed release delivery system for a nutritional composition, which provides any one or more of the above-stated effects. For the purposes of this invention, “time-released” should be understood as including any type of timed release delivery system, including but not limited to any type of quick-release delivery system, slow release delivery system and/or controlled release delivery system.

### DETAILED DESCRIPTION OF THE INVENTION

The present invention is directed to a nutritional composition that includes at least creatinol. As used herein, it is understood that the term “creatinol” also includes derivatives of creatinol such as, but not limited to, esters (e.g., ethyl ester), amides, and salts, e.g., magnesium salts, as well as other derivatives, including derivatives that become active upon metabolism. The nutritional composition according to this invention may provides at least one of: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels.

In this manner, the consumption of this nutritional composition may provide for a method of providing any one or more of: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels.

According to this invention, the nutritional composition may be consumed in any form. For instance, the dosage form of the nutritional composition may be provided as, e.g., a capsule, a tablet, a caplet, a liquid beverage, a powder beverage mix (including forms that provide an effervescent effect and those without such an effervescent effect), a dietary gel, or as a ready-to-eat bar or drink product.

Furthermore, the dosage form of the nutritional composition in accordance with this embodiment may be provided in accordance with customary processing techniques for herbal and/or dietary supplements in any of the forms mentioned above. For instance, in the embodiment in which the nutritional composition is provided as a capsule, the active ingredients may be suitably processed and encapsulated into capsules, e.g., cellulose, gelatin, etc., with suitable excipients. Those of skill in the art will appreciate that the nutritional composition may contain a variety of excipients. Still further, the nutritional composition in accordance with this embodiment may be provided in a timed release, e.g., quick-release, slow release and controlled release, delivery system. Additional details of various possible time release delivery systems are provided below.

In one embodiment of the present invention, which is set forth in greater detail in Example 1 below, there is provided a nutritional composition that includes at least creatinol O-phosphate, e.g., APLODAN™. In an embodiment, the creatinol O-phosphate, e.g., APLODAN, may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for promoting any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is tablets and/or caplets.

Furthermore, the dosage form of the nutritional composition in accordance with this embodiment may be provided in accordance with customary processing techniques for herbal and/or dietary supplements wherein the active ingredients are suitably processed and compressed into tablets and/or caplets with suitable excipients. Those of skill in the art will appreciate that the nutritional composition may contain a variety of excipients. For instance, the nutritional composition in accordance with this embodiment may have any number of different excipients, including but not limited to potassium chloride, dextrose, stearic acid, magnesium stearate, ethyl cellulose, dicalcium phosphate and color.

Still further, the nutritional composition in accordance with this embodiment may be provided in a timed release, e.g., quick release, slow release and controlled release, delivery system. The nutritional composition in accordance with this embodiment is preferably provided in a timed release, e.g., quick release, slow release and controlled release, delivery system. Additional details of various possible time release delivery systems are provided below.

In a second embodiment of the present invention, which is set forth in greater detail in Example 2 below, there is provided a nutritional composition that includes at least creatinol, creatine, alpha lipoic acid, NAC, taurine, chromium, vanadium, and ginseng. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is tablets and/or caplets.

Furthermore, the dosage form of the nutritional composition in accordance with this embodiment may be provided in accordance with customary processing techniques for herbal and/or dietary supplements wherein the active ingredients are suitably processed and compressed into tablets and/or caplets with suitable excipients. Those of skill in the art will appreciate that the nutritional composition may contain a variety of excipients. For instance, the nutritional composition in accordance with this embodiment may have any number of different excipients, including but not limited to potassium chloride, dextrose, stearic acid, magnesium stearate, ethyl cellulose, dicalcium phosphate and color.

Still further, the nutritional composition in accordance with this embodiment may be provided in a timed release, e.g., quick release, slow release and controlled release, delivery system. The nutritional composition in accordance with this embodiment is preferably provided in a timed release, e.g., quick release, slow release and controlled release, delivery system. Additional details of various possible time release delivery systems are provided below.

In a third embodiment of the present invention, which is set forth in greater detail in Example 3 below, there is provided a nutritional composition that includes at least creatinol, calcium sulfate, ciwujia, ribose, ascorbic acid, caffeine, and vanadyl sulfate. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is tablets and/or caplets.

Furthermore, the dosage form of the nutritional composition in accordance with this embodiment may be provided in accordance with customary processing techniques for herbal and/or dietary supplements wherein the active ingredients are suitably processed and compressed into tablets and/or caplets with suitable excipients. Those of skill in the art will appreciate that the nutritional composition may contain a variety of excipients. For instance, the nutritional composition in accordance with this embodiment may have any number of different excipients, including but not limited to potassium chloride, dextrose, stearic acid, magnesium stearate, ethyl cellulose, dicalcium phosphate and color.

Still further, the nutritional composition in accordance with this embodiment may be provided in a timed release, e.g., quick release, slow release and controlled release, delivery system. The nutritional composition in accordance with this embodiment is preferably provided in a timed release, e.g., quick release, slow release and controlled release, delivery system. Additional details of various possible time release delivery systems are provided below.

In a fourth embodiment of the present invention, which is set forth in greater detail in Example 4 below, there is provided a nutritional composition that includes at least creatinol, oral ATP, arginine, citrulline malate, pinitol, hydroxymethyl butyrate (HMB), and vinpocetine. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is tablets and/or caplets.

Furthermore, the dosage form of the nutritional composition in accordance with this embodiment may be provided in accordance with customary processing techniques for herbal and/or dietary supplements wherein the active ingredients are suitably processed and compressed into tablets and/or caplets with suitable excipients. Those of skill in the art will appreciate that the nutritional composition may contain a variety of excipients. For instance, the nutritional composition in accordance with this embodiment may have any number of different excipients, including but not limited to potassium chloride, dextrose, stearic acid, magnesium stearate, ethyl cellulose, dicalcium phosphate and color.

Still further, the nutritional composition in accordance with this embodiment may be provided in a timed release, e.g., quick release, slow release and controlled release, delivery system. The nutritional composition in accordance with this embodiment is preferably provided in a timed release, e.g., quick release, slow release and controlled release, delivery system. Additional details of various possible time release delivery systems are provided below.

In a fifth embodiment of the present invention, which is set forth in greater detail in Example 5 below, there is provided a nutritional composition that includes at least creatinol, ALA, evodiamine, yohimbine HCl, niacin, and caffeine. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is capsules.

Furthermore, the dosage form of the nutritional composition in accordance with this embodiment may be provided in accordance with customary processing techniques for herbal and/or dietary supplements wherein the active ingredients are suitably processed and encapsulated into capsules, e.g., cellulose, gelatin, etc., with suitable excipients. Those of skill in the art will appreciate that the nutritional composition may contain a variety of excipients. For instance, the nutritional composition in accordance with this embodiment may have any number of different excipients, including but not limited to gelatin, microcrystalline cellulose, magnesium stearate, and silicon dioxide.

In a sixth embodiment of the present invention, which is set forth in greater detail in Example 6 below, there is provided a nutritional composition that includes at least creatinol, thiamine, riboflavin, vitamin B6, zinc, magnesium, selenium, and conjugated linoleic acid (CLA). In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is capsules.

Furthermore, the dosage form of the nutritional composition in accordance with this embodiment may be provided in accordance with customary processing techniques for herbal and/or dietary supplements wherein the active ingredients are suitably processed and encapsulated into capsules, e.g., cellulose, gelatin, etc., with suitable excipients. Those of skill in the art will appreciate that the nutritional composition may contain a variety of excipients. For instance, the nutritional composition in accordance with this embodiment may have any number of different excipients, including but not limited to gelatin, microcrystalline cellulose, magnesium stearate, and silicon dioxide.

In a seventh embodiment of the present invention, which is set forth in greater detail in Example 7 below, there is provided a nutritional composition that includes at least creatinol, caffeine, HMB, zinc, chromium, pycnogenol, and alpha lipoic acid (ALA). In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is capsules.

Furthermore, the dosage form of the nutritional composition in accordance with this embodiment may be provided in accordance with customary processing techniques for herbal and/or dietary supplements wherein the active ingredients are suitably processed and encapsulated into capsules, e.g., cellulose, gelatin, etc., with suitable excipients. Those of skill in the art will appreciate that the nutritional composition may contain a variety of excipients. For instance, the nutritional composition in accordance with this embodiment may have any number of different excipients, including but not limited to gelatin, microcrystalline cellulose, magnesium stearate, and silicon dioxide.

In an eighth embodiment of the present invention, which is set forth in greater detail in Example 8 below, there is provided a nutritional composition that includes at least creatinol, calcium pyruvate, sodium bicarbonate, alpha lipoic acid, chromium, citrulline malate, vitamin E, vitamin C, and glycerol. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is capsules.

Furthermore, the dosage form of the nutritional composition in accordance with this embodiment may be provided in accordance with customary processing techniques for herbal and/or dietary supplements wherein the active ingredients are suitably processed and encapsulated into capsules, e.g., cellulose, gelatin, etc., with suitable excipients. Those of skill in the art will appreciate that the nutritional composition may contain a variety of excipients. For instance, the nutritional composition in accordance with this embodiment may have any number of different excipients, including but not limited to silica, cellulose, gelatin, and magnesium stearate.

In a ninth embodiment of the present invention, which is set forth in greater detail in Example 9 below, there is provided a nutritional composition that includes at least creatinol, DHEA, betaine, 3,3′-diindolylmethane (DIM), eurycoma longofolia, tribulus, and caffeine. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is capsules.

Furthermore, the dosage form of the nutritional composition in accordance with this embodiment may be provided in accordance with customary processing techniques for herbal and/or dietary supplements wherein the active ingredients are suitably processed and encapsulated into capsules, e.g., cellulose, gelatin, etc., with suitable excipients. Those of skill in the art will appreciate that the nutritional composition may contain a variety of excipients. For instance, the nutritional composition in accordance with this embodiment may have any number of different excipients, including but not limited to gelatin, magnesium stearate, silica, and cellulose.

In a tenth embodiment of the present invention, which is set forth in greater detail in Example 10 below, there is provided a nutritional composition that includes at least creatinol, dextrose, whey protein, branched chain amino acids (BCAAs), alpha lipoic acid, glucosamine, chondroitin sulfate, policosanol, and phosphatidylserine. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is powder beverage mix (including forms that provide an effervescent effect and those without such an effervescent effect).

Those of skill in the art will appreciate that the nutritional composition may contain a variety of excipients. For instance, the nutritional composition in accordance with this embodiment may have any number of different excipients, including but not limited to calcium silicate and natural/artificial flavors.

In an eleventh embodiment of the present invention, which is set forth in greater detail in Example 11 below, there is provided a nutritional composition that includes at least creatinol and carnosine. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is powder beverage mix (including forms that provide an effervescent effect and those without such an effervescent effect).

Those of skill in the art will appreciate that the nutritional composition may contain a variety of excipients. For instance, the nutritional composition in accordance with this embodiment may have any number of different excipients, including but not limited to calcium silicate and natural/artificial flavors.

In a twelfth embodiment of the present invention, which is set forth in greater detail in Example 12 below, there is provided a nutritional composition that includes at least creatinol and corosolic acid. In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is powder beverage mix (including forms that provide an effervescent effect and those without such an effervescent effect).

Those of skill in the art will appreciate that the nutritional composition may contain a variety of excipients. For instance, the nutritional composition in accordance with this embodiment may have any number of different excipients, including but not limited to calcium silicate and natural/artificial flavors.

In a thirteenth embodiment of the present invention, which is set forth in greater detail in Example 13 below, there is provided a nutritional composition that includes at least creatinol O-phosphate (Aplodan™). In an embodiment, the creatinol may be fine-milled, which is described in greater detail below. The nutritional composition according to this embodiment, provides at least one of the following benefits: strong performance enhancing effect with no loading needed; push muscle past normal working abilities, e.g., past normal limits; enable more time under stress, e.g., 83% more time under stress in only 7 days; protect muscle fibers to allow the user to withstand more mechanical stress; buffers lactic acid for more stable pH in muscle; acts as an H+ (hydrogen ion) blocker; controls pH of muscles, e.g., controls pH decline; controls excess hydrogen ions; acts as a lactic acid controller; better lifting capacity; enables effective use of overload principle; buffers at the muscle fiber level; withstand more stimulus (stimuli); forces muscle adaptation for greater size and strength; increase muscle fiber performance; prolongs time under tension; enables a user to withstand more pain; buffers lactic acid for more stable pH in muscle; better lifting capacity; maximum peak muscle contraction and power output. In addition, the nutritional composition may enable a user to one or more of: increase lean mass; increase total body mass; and increase muscle growth/size. Still further, the nutritional composition may enable a user to one or more of: increase cardiovascular performance; support cardiovascular health; increases oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels. In this manner, the consumption of this nutritional composition provides for a method for any one or more of the above-stated effects.

According to this embodiment, the nutritional composition may be provided and consumed in any form, e.g., a capsule, a tablet, a caplet, a liquid beverage, a powder beverage mix (including forms that provide an effervescent effect and those without such an effervescent effect), a dietary gel, or as a ready-to-eat bar or drink product. However, the preferred form for consumption of the nutritional composition in accordance with this embodiment is powder beverage mix (including forms that provide an effervescent effect and those without such an effervescent effect).

Those of skill in the art will appreciate that the nutritional composition may contain a variety of excipients. For instance, the nutritional composition in accordance with this embodiment may have any number of different excipients, including but not limited to calcium silicate and natural/artificial flavors.

Creatinol

As used herein, “creatinol” may refer to the chemical compound N-methyl-N-(beta-hydroxyethyl) guanidine O-phosphate (creatinol O-phosphate, COP, and also known as APLODAN™), CAS Registry No. 6903-80-6, also known as creatinol-phosphate. The term “creatinol” may also refer to creatinol-sulfate and other derivatives.

Although the present invention is not to be limited by any theoretical explanation, it is believed that creatinol-phosphate is involved in and enhances ATP synthesis, which is reduced during muscular exercise. Creatinol-phosphate has been shown to increase muscular strength in human subjects, which is also marked in the elderly, as well as reduce asthenia and muscular weakness in convalescent subjects. It is believed that creatinol-phosphate allows for more endurance in the form of muscular contraction before fatigue sets in.

As used herein, a serving of the nutritional composition comprises from about 0.1 g to about 10 g of creatinol-phosphate. More preferably, a serving of the nutritional composition comprises from about 0.5 g to about 5 g of creatinol-phosphate, and most preferably about 1.5 grams, twice daily.

Creatine

As used herein, “creatine” refers to the chemical compound N-methyl-N-guanyl glycine, (CAS Registry No. 57-00-1), also known as, (α-methyl guanido) acetic acid, N-(aminoiminomethyl)-N-glycine, and methylglycocyamine, and Methylguanidoacetic acid, and N-Methyl-N-guanylglycine. As used herein, “creatine” also includes derivatives of creatine such as esters, and amides, and salts, as well as other derivatives, including derivatives that become active upon metabolism. The chemical structure of creatine is as follows:

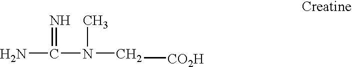

While not wishing to be bound by theory it is believed that creatine increases strength and muscle size as well as cell volumization.

Creatine and creatine derivatives are widely available from a number of commercial sources. Commercially available creatine derivatives include creatine phosphate, creatine citrate, magnesium creatine, alkaline creatine, creatine pyruvate, creatine hydrates (including, but not limited to creatine monohydrate), di-creatine malate and tri-creatine malate. Glycocyamine, an in vivo precursor of creatine, is also commercially available and suitable in the practice of the present invention. The nutritional composition preferably comprises tri-creatine malate and creatine monohydrate.

As used herein, a serving of the nutritional composition comprises from about 0.1 g to about 10 g of creatine. More preferably, a serving of the nutritional composition comprises from about 0.5 g to about 5 g of creatine. Most preferably, a serving of the nutritional composition comprises about 1.5 g of creatine twice daily.

Alpha Lipoic Acid

As used herein, “alpha lipoic acid” (ALA) preferably refers to the chemical compound 1,2-dithiolane-3-pentanoic acid, CAS registry No. 62-46-4, also known as, thioctic acid and 6,8-dithio octanoic acid. As used herein, “alpha lipoic acid” also includes derivatives of alpha lipoic acid such as esters, and amides, as well as other derivatives, including derivatives that become active upon metabolism. The chemical structure of alpha lipoic acid is as follows:

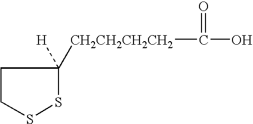

Alpha lipoic acid is an insulin modulator and an antioxidant that serves as protection against oxidative injury in non-neuronal and neuronal tissue. Alpha lipoic acid is a nutrient that the human body makes in minute quantities and may be obtained from yeast and liver. Studies have shown that alpha lipoic acid can significantly increase the body's utilization of blood sugar in type II diabetics and that lipoic acid may increase the metabolic clearance rate of glucose by 50% in diabetics. In Europe, alpha lipoic acid has been used as a substitute for insulin in the treatment of Type II diabetes.

Although the present invention is not to be limited by any theoretical explanation, it is believed that insulin is a primary factor that stimulates glucose and creatine transport into the muscle cells and that alpha lipoic acid both mimics and enhances the actions of insulin in glucose and creatine transport into the muscle cells.

Preferably, a serving of the nutritional composition comprises from about 0.1 mg to about 10 mg of alpha lipoic acid per gram of nutritional composition. More preferably, a serving of the nutritional composition comprises from about 0.5 mg to about 5 mg of alpha lipoic acid per gram of nutritional composition.

N-Acetyl-Cysteine

N-acetyl cysteine (NAC) is the N-acetyl derivative of the amino acid L-cysteine. N-acetyl cysteine is a precursor of glutathione, which is a potent antioxidant. Glutathione can not cross the cell membrane, but N-acetyl cysteine easily crosses the cell membrane where it is converted to cysteine and, subsequently, glutathione. Reactive oxygen species (ROS) such as hydrogen peroxide and hydroxyl-free radicals reduce intracellular and extracellular concentrations of glutathione. N-acetyl cysteine is an efficient way to replenish glutathione and reduce damage caused by ROS.

L-taurine

L-taurine is an amino acid that is not involved in the synthesis of proteins in animals and is the end product of L-cysteine metabolism. L-taurine is the principle free intracellular amino acid found in human tissue. L-taurine is also an antioxidant, and has been shown to improve insulin sensitivity.

Chromium

Chromium has been shown to improve insulin sensitivity and glucose disposal. Chromium is supplied as a chromium chelate. Preferred chromium chelate includes chromium picolinate and chromium nicotinate. Most preferred chromium chelate is chromium nicotinate or polynicotinate.

Vanadium (Vanadyl Sulfate)

Vanadium is a trace mineral. Vanadium appears to be important in normal bone growth and as a cofactor for various enzyme reactions. Vanadium inhibits ATPases, phosphatases, and phosphoryl-transfer enzymes. Some evidence suggests that vanadium can mimic the actions of insulin, possibly by causing phosphorylation of insulin receptor proteins. Vanadium activates the receptor; stimulates glucose oxidation and transport; inhibits lipolysis in adipose tissue; stimulates glycogen synthesis in the liver; inhibits hepatic gluconeogenesis; inhibits intestinal glucose transport; and increases glucose uptake, utilization, and glycogen synthesis in skeletal muscle.

Ginseng

As used herein, Ginseng preferably refers to American Ginseng (Panax quinquefolium L.), or Korean Ginseng (Panax Ginseng) or Siberian Ginseng ((Eleutherococcus senticosus) and derivatives thereof. Most preferably Ginseng refers to American Ginseng (Panax quinquefolium L.), or Korean Ginseng (Panax Ginseng) and derivatives thereof.

While not being bound by theory it is believed that Ginseng stimulates the production of nitric oxide.

Timed Release Delivery System

The present invention also provides for a timed release delivery system for a nutritional composition for providing any of the above-mentioned effects, which comprises a combination of pharmaceutical grade excipients comprising Hydroxypropyl Methyl Cellulose (Methocellulose K100M Premium CR EP-Hypromellose 2208 USP XXII), Ethyl Cellulose (Ethocellulose Standard FP Premium—NF XVII), and Hydroxypropylcellulose (Klucel LF—NF XVII).

The present invention also provides for a timed release delivery system for providing any of the above-mentioned effects, which comprises a hydrophilic caplet matrix system comprising water-soluble (i.e., Hydroxypropyl Methyl Cellulose and Hydroxypropylcellulose) and water-insoluble (i.e., Ethyl Cellulose) polymers uniformly incorporated at a specified ratio.

In a timed release delivery system for a nutritional composition for providing any of the above-mentioned effects, the hydrophilic caplet matrix system is in the ratio of 1 Hydroxypropylcellulose: 1 Ethyl Cellulose: 0.5 Hydroxypropyl Methyl Cellulose.

According to an embodiment of the invention, the timed release feature may provide a hydrocolloid-coated gel-forming fiber. This caplet containing the dietary ingredients provides a unique, efficient and controllable prolonged-action drug-delivery system. The solid prolonged (timed/extended) release technology consists of an effective dose of particles of a biologically-absorbable dietary ingredients, gel-forming dietary matrix particles, a mineral salt of carbonate or bicarbonate which releases a physiologically-acceptable gas upon ingestion, and a physiologically-acceptable acid, the combination of the fiber and mineral salt providing amounts which effect prolonged but effective release of the dietary ingredients upon oral ingestion and exposure of the caplets to biological fluids.

The gel-forming dietary fiber may comprise about 35% to about 50% by weight, the mineral salt may comprise about 5% to about 75% by weight, the physiologically-acceptable acid may comprise 0% to about 50% by weight, and the ratio of the weight of the gel-forming fiber to the weight of the drug or therapeutic agent may be between about 1,000:0.5 and 1:1.5, the gel-forming dietary fiber particles being coated with a hydrocolloid film and the dietary ingredients being optionally coated with a film of the same or a different hydrocolloid.

By precoating the gel-forming fiber or gum particles, and optionally but advantageously also the dietary ingredients, with a film of Sodium Carboxymethylcellulose (NACMC) or other hydrocolloid, including hydrocolloids such as, e.g., natural and modified gums, celluloses and modified celluloses, pectin, mucillages, modified starches, noncellulosic polysaccharides, algal polysaccharides, and mixtures thereof, particularly preferred hydrocolloids including carboxymethyl cellulose, methyl cellulose, karaya gum, acacia gum, sodium alginate, calcium alginate, hydroxypropylmethylcellulose, and mixtures thereof, and then tableting or granulating together with a mineral carbonate or bicarbonate to speed up hydration, the locking up or sealing from further hydration can be prevented and a smooth and controlled release of the dietary supplement can be achieved.

Compositions that include such an extended release delivery system may prolong the release of dietary ingredients upon administration to a user. Such a composition may work up to 10 to 12 hours after ingestion i.e., the dietary ingredients being released and staying in the blood stream for several hours.

Although the following examples illustrate the practice of the present invention in some of its embodiments, the examples should not be construed as limiting the scope of the invention. Other embodiments will be apparent to one skilled in the art from consideration of the specification and examples.

### EXAMPLE 1

A serving of the nutritional composition comprises the following ingredients.

| Ingredient #: | Ingredients |
|---|---|
| 1 | Creatinol |

### EXAMPLE 2

A serving of the nutritional composition comprises the following ingredients.

| Ingredient #: | Ingredients |
|---|---|
| 1 | Creatinol |
| 2 | Creatine |
| 3 | Alpha Lipoic Acid |
| 4 | NAC |
| 5 | Taurine |
| 6 | Chromium |
| 7 | Vanadium |
| 8 | Ginseng |

### EXAMPLE 3

A serving of the nutritional composition comprises the following ingredients.

| Ingredient #: | Ingredients |
|---|---|
| 1 | Creatinol |
| 2 | Calcium Sulfate |
| 3 | Ciwuja |
| 4 | Ribose |
| 5 | Ascorbic Acid |
| 6 | Caffeine |
| 7 | Vanadyl Sulfate |

### EXAMPLE 4

A serving of the nutritional composition comprises the following ingredients.

| Ingredient #: | Ingredients |
|---|---|
| 1 | Creatinol |
| 2 | Oral ATP |
| 3 | Arginine (BCAA's) |
| 4 | Citrulline Malate |
| 5 | Pinitol |
| 6 | HMB |
| 7 | Vinpocetine |

### EXAMPLE 5

A serving of the nutritional composition comprises the following ingredients.

| Ingredient #: | Ingredients |
|---|---|
| 1 | Creatinol |
| 2 | ALA |
| 3 | Evodiamine |
| 4 | Yohimbine HCL |
| 5 | Niacin |
| 6 | Caffeine |

### EXAMPLE 6

A serving of the nutritional composition comprises the following ingredients.

| Ingredient #: | Ingredients |
|---|---|
| 1 | Creatinol |
| 2 | Thiamine |
| 3 | Riboflavin |
| 4 | Vitamin B6 |
| 5 | Zinc |
| 6 | Magnesium |
| 7 | Selenium |
| 8 | CLA |

### EXAMPLE 7

A serving of the nutritional composition comprises the following ingredients.

| Ingredient #: | Ingredients |
|---|---|
| 1 | Creatinol |
| 2 | Caffeine |
| 3 | HMB |
| 4 | Zinc |
| 5 | Chromium |
| 6 | Pycnogenol |
| 7 | ALA |

### EXAMPLE 8

A serving of the nutritional composition comprises the following ingredients.

| Ingredient #: | Ingredients |
|---|---|
| 1 | Creatinol |
| 2 | Calcium Pyruvate |
| 3 | Sodium Bicarbonate |
| 4 | Alpha Lipoic Acid |
| 5 | Chromium |
| 6 | Citrulline Malate |
| 7 | Vitamin E |
| 8 | Vitamin C |
| 9 | Glycerol |

### EXAMPLE 9

A serving of the nutritional composition comprises the following ingredients.

| Ingredient #: | Ingredients |
|---|---|
| 1 | Creatinol |
| 2 | DHEA |
| 3 | Betaine |
| 4 | DIM |
| 5 | Eurycoma Longofolia |
| 6 | Tribulus |
| 7 | Caffeine |

### EXAMPLE 10

A serving of the nutritional composition comprises the following ingredients.

| Ingredient #: | Ingredients |
|---|---|
| 1 | Creatinol |
| 2 | Maltodextrine |
| 3 | Whey Protein |
| 4 | BCAA's |
| 5 | Alpha Lipoic Acid |
| 6 | Glucosamine |
| 7 | Chondroitin Sulfate |
| 8 | Policosanol |
| 9 | Phosphatidylserine |

### EXAMPLE 11

A serving of the nutritional composition comprises the following ingredients.

| Ingredient #: | Ingredients |
|---|---|
| 1 | Creatinol |
| 2 | Carnosine |

### EXAMPLE 12

A serving of the nutritional composition comprises the following ingredients.

| Ingredient #: | Ingredients |
|---|---|
| 1 | Creatinol |
| 2 | Corosolic Acid |

### EXAMPLE 13

A 1.5 gram serving of the nutritional composition, ingested twice daily, the following ingredients.

| Ingredient #: | Ingredients |
|---|---|
| 1 | Creatinol O-phosphate (Aplodan ™) |

What is claimed is:

1. A nutritional composition for one of promoting muscle performance and acting as a hydrogen (H+) blocker, the nutritional composition comprising at least one of a salt, ester or amide of creatinol.

2. The nutritional composition of claim 1, wherein the creatinol is creatinol O-phosphate.

3. The nutritional composition of claim 1, further comprising creatine, alpha lipoic acid, NAC, taurine, chromium, vanadium, and ginseng.

4. The nutritional composition of claim 1, further comprising calcium sulfate, ciwujia, ribose, ascorbic acid, caffeine, and vanadyl sulfate.

5. The nutritional composition of claim 1, further comprising oral ATP, arginine, citrulline malate, pinitol, hydroxymethyl butyrate (HMB), and vinpocetine.

6. The nutritional composition of claim 1, further comprising ALA, evodiamine, yohimbine HCl, niacin, and caffeine.

7. The nutritional composition of claim 1, further comprising thiamine, riboflavin, vitamin B6, zinc, magnesium, selenium, and conjugated linoleic acid (CLA).

8. The nutritional composition of claim 1, further comprising caffeine, HMB, zinc, chromium, pycnogenol, and alpha lipoic acid (ALA).

9. The nutritional composition of claim 1, further comprising calcium pyruvate, sodium bicarbonate, alpha lipoic acid, chromium, citrulline malate, vitamin E, vitamin C, and glycerol.

10. The nutritional composition of claim 1, further comprising DHEA, betaine, 3,3′-diindolylmethane (DIM), eurycoma longofolia, tribulus, and caffeine.

11. The nutritional composition of claim 1, further comprising dextrose, whey protein, branched chain amino acids (BCAAs), alpha lipoic acid, glucosamine, chondroitin sulfate, policosanol, and phosphatidylserine.

12. The nutritional composition of claim 1, further comprising carnosine.

13. The nutritional composition of claim 1, further comprising corosolic acid.

14. The nutritional composition of claim 1, wherein the creatinol is present in an amount effective to at least one of increase cardiovascular performance; support cardiovascular health; increase oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels.

15. The nutritional composition of claim 1, wherein the creatinol is present in an amount effective to provide at least one of the following benefits: strong performance enhancing effect with no loading needed; push muscle past normal working abilities, e.g., past normal limits; enable more time under stress; protect muscle fibers to allow a user to withstand more mechanical stress; buffers lactic acid for more stable pH in muscle; act as an H+ (hydrogen ion) blocker; controls pH of muscles; controls excess hydrogen ions; acts as a lactic acid controller; better lifting capacity; enable effective use of overload principle; buffer at the muscle fiber level; withstand more stimulus (stimuli); forces muscle adaptation for greater size and strength; increase muscle fiber performance; prolong time under tension; enable a user to withstand more pain; buffer lactic acid for more stable pH in muscle; better lifting capacity; maximum peak muscle contraction and power output.

16. The nutritional composition of claim 1, wherein the creatinol is present in an amount effective to provide an improvement in the symptoms associated with amyotrophic lateral sclerosis.

17. The nutritional composition of claim 1, wherein the nutritional composition includes a timed release delivery system and wherein the timed release delivery system is one of quick release, slow release and controlled release.

18. A method for one of promoting muscle performance and acting as a hydrogen (H+) blocker, comprising ingesting a nutritional composition comprising at least one of a salt, ester or amide of creatinol.

19. The method of claim 18, wherein the creatinol is creatinol O-phosphate.

20. The method of claim 18, wherein the nutritional composition further comprises creatine, alpha lipoic acid, NAC, taurine, chromium, vanadium, and ginseng.

21. The method of claim 18, wherein the nutritional composition further comprises calcium sulfate, ciwujia, ribose, ascorbic acid, caffeine, and vanadyl sulfate.

22. The method of claim 18, wherein the nutritional composition further comprises oral ATP, arginine, citrulline malate, pinitol, hydroxymethyl butyrate (HMB), and vinpocetine.

23. The method of claim 18, wherein the nutritional composition further comprises ALA, evodiamine, yohimbine HCl, niacin, and caffeine.

24. The method of claim 18, wherein the nutritional composition further comprises thiamine, riboflavin, vitamin B6, zinc, magnesium, selenium, and conjugated linoleic acid (CLA).

25. The method of claim 18, wherein the nutritional composition further comprises caffeine, HMB, zinc, chromium, pycnogenol, and alpha lipoic acid (ALA).

26. The method of claim 18, wherein the nutritional composition further comprises calcium pyruvate, sodium bicarbonate, alpha lipoic acid, chromium, citrulline malate, vitamin E, vitamin C, and glycerol.

27. The method of claim 18, wherein the nutritional composition further comprises DHEA, betaine, 3,3′-diindolylmethane (DIM), eurycoma longofolia, tribulus, and caffeine.

28. The method of claim 18, wherein the nutritional composition further comprises dextrose, whey protein, branched chain amino acids (BCAAs), alpha lipoic acid, glucosamine, chondroitin sulfate, policosanol, and phosphatidylserine.

29. The method of claim 18, wherein the nutritional composition further comprises carnosine.

30. The method of claim 18, wherein the nutritional composition further comprises corosolic acid.

31. The method of claim 18, wherein the creatinol is present in an amount effective to one of increase cardiovascular performance; support cardiovascular health; increase oxygen availability; increase endurance; increase work capacity; increase strength; increase time to exhaustion; increase aerobic and anaerobic capacity and/or performance; increase muscular performance; increase oxygen availability; increase workout intensity; increase muscle size; decrease muscle soreness; and increase muscle creatinol levels.

32. The method of claim 18, wherein the creatinol is present in an amount effective to provide at least one of the following benefits: strong performance enhancing effect with no loading needed; push muscle past normal working abilities, e.g., past normal limits; enable more time under stress; protect muscle fibers to allow a user to withstand more mechanical stress; buffers lactic acid for more stable pH in muscle; act as an H+ (hydrogen ion) blocker; controls pH of muscles; controls excess hydrogen ions; acts as a lactic acid controller; better lifting capacity; enable effective use of overload principle; buffer at the muscle fiber level; withstand more stimulus (stimuli); forces muscle adaptation for greater size and strength; increase muscle fiber performance; prolong time under tension; enable a user to withstand more pain; buffer lactic acid for more stable pH in muscle; better lifting capacity; maximum peak muscle contraction and power output.

33. The method of claim 18, wherein the creatinol is present in an amount effective to provide an improvement in the symptoms associated with amyotrophic lateral sclerosis.

34. The method of claim 18, wherein the nutritional composition includes a timed release delivery system and wherein the timed release delivery system is one of quick release, slow release and controlled release.
